(19) United States
(12) Patent Application Publication
(10) Pub. No.: US 2026/0152458 A1
(43) Pub. Date: Jun. 4, 2026

(54) COMPOUND, ION CONDUCTIVE POLYMER MANUFACTURED BY THE SAME AND SEPARATOR INCLUDING THE SAME

(71) Applicant: SK INNOVATION CO., LTD., Seoul (KR)

(72) Inventors: Cheol Woo KIM, Daejeon (KR); Dong Gun LEE, Daejeon (KR); So Young LEE, Daejeon (KR); Soo Min CHO, Daejeon (KR); Jong Chan KIM, Daejeon (KR); Jung A LEE, Daejeon (KR)

(21) Appl. No.: 19/243,011

(22) Filed: Jun. 19, 2025

(30) Foreign Application Priority Data
Dec. 3, 2024 (KR) 10-2024-0177586

(51) Int. Cl. C07C 41/30; C08F 12/32; C25B 13/08; H01M 8/0239; H01M 50/414

(52) U.S. Cl. CPC C07C 41/30; C08F 12/32; C25B 13/08; H01M 8/0239; H01M 50/414

(57) ABSTRACT

Embodiments of the present disclosure provide a compound including an organic group in which at least one hydrogen is substituted with a halogen atom, an ether group and a multi-aromatic ring, which is represented by Formula 1 below,

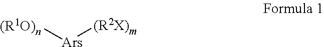

wherein in Formula 1, m and n are each independently an integer of 1 to 6, R¹ is each independently an organic group having 1 to 10 carbon atoms, R² is each independently an organic group having 1 to 20 carbon atoms, which includes at least one of an alkylene group or an arylene group, X is a halogen atom, and Ars is a multi-aromatic ring having 10 to 50 carbon atoms.

### CROSS-REFERENCE TO RELATED APPLICATIONS

This patent application claims the priority and benefits of Korean Patent Application No. 10-2024-0177586 filed on Dec. 3, 2024, the disclosure of which is incorporated herein by reference in its entirety.

### BACKGROUND

### 1. Field of the Invention

The embodiments of the present disclosure relate to a compound, an ion conductive polymer manufactured by the compound and a separator including the ion conductive polymer.

### 2. Description of the Related Art

Generally, a separator is a barrier which exists between two different substances, and allows a specific substance to selectively pass therethrough. The separators may be divided into various types according to properties, materials, shapes, etc. thereof. An ion exchange membrane is a type of separator that is a single membrane that is homogeneous, non-porous, symmetrical, hydrophilic, carries a charge, and may serve as a liquid separator and the like.

The ion exchange membrane is a membrane having an ion exchange function for a specific ion, which is a type of high efficiency separator. When introducing an ion exchange membrane into an electrolyte solution and applying a current thereto, functional groups having positive or negative charges on pore walls of the membrane attract only specific ions having opposite charges into the pores of the membrane, thereby retaining electrical neutrality. The specific ions that have been bound to the functional groups of the membrane pass through the membrane by continuously repeating the binding and dissociation with the functional groups.

The ion exchange membranes may be classified into a cation exchange membrane (CEM), an anion exchange membrane (AEM), and a bipolar exchange membrane (BEM) according to characteristics thereof. The cation exchange membrane is a membrane which allows only cations to pass therethrough while anions serve as the fixed charges. The anion exchange membrane is a membrane which allows only anions to pass therethrough while cations serve as the fixed charges. The bipolar exchange membrane has a form in which cation and anion exchange membranes are coupled to both sides thereof, and allows both the cations and anions to selectively pass therethrough.

The ion exchange membrane may be used in various fields such as water treatment, chemistry, energy industries and the like. Generally, ion exchange membranes allow different specific charges to selectively pass therethrough, and exhibit low electrical resistance and high ionic conductivity. In addition, ion exchange membranes may possess excellent mechanical strength and chemical stability in order to ensure continuous, long-term use.

### SUMMARY

An embodiments of the present disclosure provide a compound for manufacturing an ion conductive polymer having improved electrochemical properties.

Another embodiment of the present disclosure provides an ion conductive polymer and a separator manufactured from the compound.

According to embodiments of the present disclosure, there is provided a compound represented by Formula 1 below.

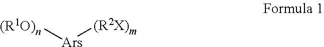

In Formula 1, m and n are each independently an integer of 1 to 6, R¹ is each independently an organic group having 1 to 10 carbon atoms, R² is each independently an organic group having 1 to 20 carbon atoms, R² includes at least one of an alkylene group or an arylene group, X is a halogen atom, and Ars is a multi-aromatic ring having 10 to 50 carbon atoms.

According to an embodiment, the compound represented by Formula 1 above may include at least one of compounds represented by Formulas 2 to 4 below.

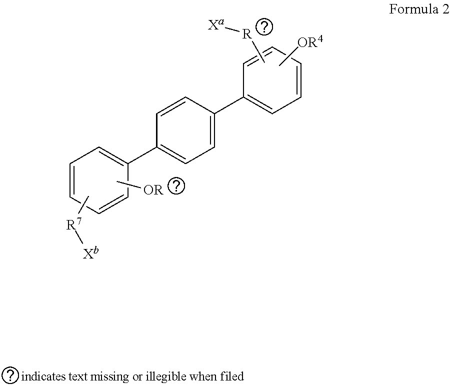

In Formula 2, R⁴ and R⁵ are each independently an organic group having 1 to 10 carbon atoms, R⁶ and R⁷ are each independently an organic group having 1 to 20 carbon atoms, R⁶ and R⁷ include at least one of an alkylene group or an arylene group, and X^{a} and X^{b} are halogen atoms.

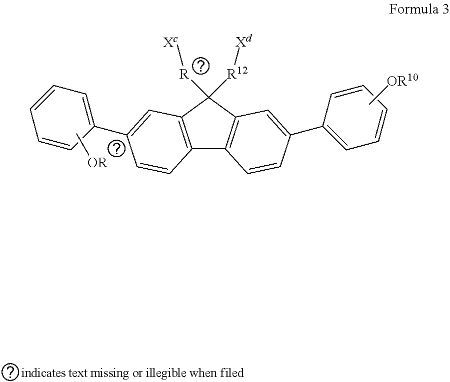

In Formula 3, R⁹ and R¹⁰ are each independently an organic group having 1 to 10 carbon atoms, R¹¹ and R¹² are each independently an organic group having 1 to 20 carbon atoms, R¹¹ and R¹² include at least one of an alkylene group or an arylene group, and X^{c} and X^{d} are halogen atoms.

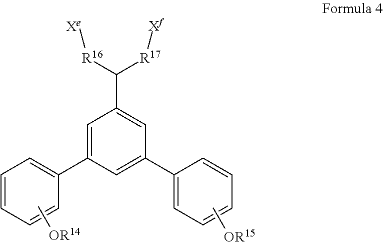

In Formula 4, R¹⁴ and R¹⁵ are each independently an organic group having 1 to 10 carbon atoms, R¹⁶ and R¹⁷ are each independently an organic group having 1 to 20 carbon atoms, R¹⁶ and R¹⁷ include at least one of an alkylene group or an arylene group, and X^{e} and X^{f} are halogen atoms.

According to an embodiment, the compounds represented by Formulas 2 to 4 above may include compounds represented by Formulas 2-1 to 4-1 below, respectively.

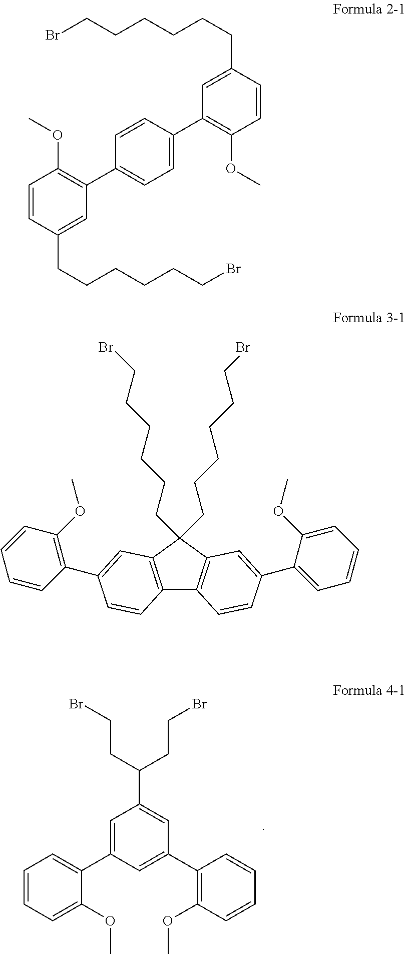 [SMILES: COc1ccc(CCCCCCBr)cc1-c1ccc(-c2cc(CCCCCCBr)ccc2OC)cc1 ; COc1ccccc1-c1ccc2c(c1)C(CCCCCCBr)(CCCCCCBr)c1cc(-c3ccccc3OC)ccc1-2 ; COc1ccccc1-c1cc(-c2ccccc2OC)cc(C(CCBr)CCBr)c1]

According to an embodiment, the compound may be a monomer for polymerization.

In addition, according to another embodiment of the present disclosure, there is provided anion conductive polymer including a repeating unit represented by Formula 5 below.

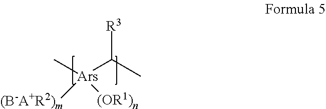

In Formula 5, A⁺ is a quaternary ammonium, B⁻ is an anion, m and n are each independently an integer of 1 to 6, R¹ is each independently an organic group having 1 to 10 carbon atoms, R² is each independently an organic group having 1 to 20 carbon atoms, R² includes at least one of an alkylene group or an arylene group, R³ is H or an organic group having 1 to 15 carbon atoms, and Ars is a multi-aromatic ring having 10 to 50 carbon atoms.

According to an embodiment, the repeating unit represented by Formula 5 above may include at least one of repeating units represented by Formulas 6 to 8 below.

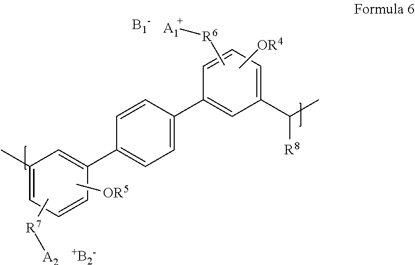

In Formula 6, A₁⁺ and A₂⁺ are quaternary ammoniums, B₁⁻ and B₂⁻ are anions, R⁴ and R⁵ are each independently an organic group having 1 to 10 carbon atoms, R⁶ and R⁷ are each independently an organic group having 1 to 20 carbon atoms, R⁶ and R⁷ include at least one of an alkylene group or an arylene group, and R⁸ is H or an organic group having 1 to 15 carbon atoms.

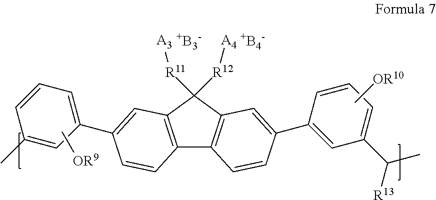

In Formula 7, A₃⁺ and A₄⁺ are quaternary ammoniums, B₃⁻; and B₄⁻ are anions, R⁹ and R¹⁰ are each independently an organic group having 1 to 10 carbon atoms, R¹¹ and R¹² are each independently an organic group having 1 to 20 carbon atoms, R¹¹ and R¹² include at least one of an alkylene group or an arylene group, and R¹³ is H or an organic group having 1 to 15 carbon atoms.

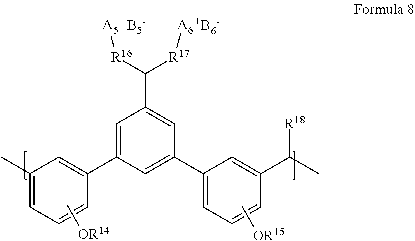

In Formula 8, A₅⁺ and A₆⁺ are quaternary ammoniums, B₅⁻ and B₆⁻ are anions, R¹⁴ and R¹⁵ are each independently an organic group having 1 to 10 carbon atoms, R¹⁶ and R¹⁷ are each independently an organic group having 1 to 20 carbon atoms, R¹⁶ and R¹⁷ include at least one of an alkylene group or an arylene group, and R¹⁸ is H or an organic group having 1 to 15 carbon atoms.

According to an embodiment, the repeating units represented by Formulas 6 to 8 above may include repeating units represented by Formulas 6-1 to 8-1 below, respectively.

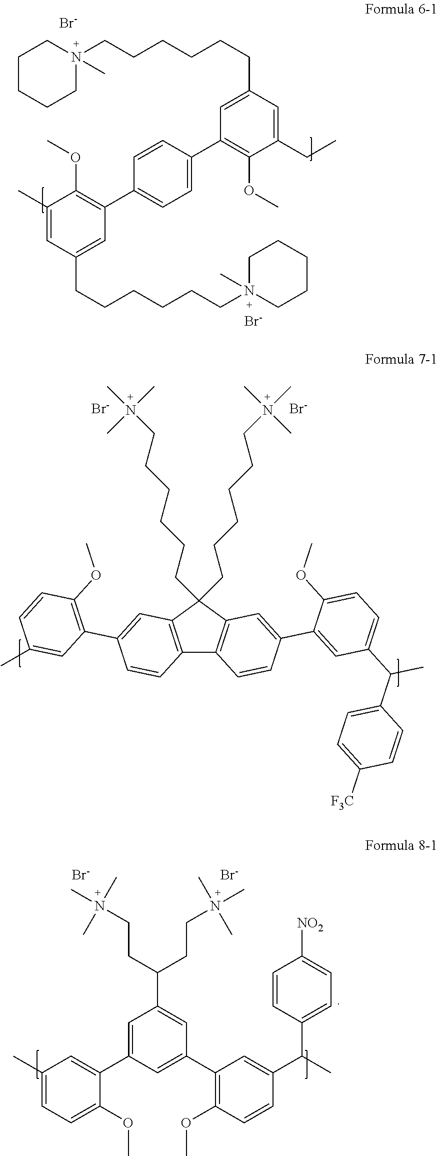

According to an embodiment, an OH ionic conductivity of the ion conductive polymer at 25° C. may be 40 mS/cm (millisiemens per centimeter) to 80 mS/cm.

Furthermore, according to another embodiment of the present disclosure, there is provided a device including a cathode; an anode disposed to face the cathode; and the above-described separator. According to an embodiment, the device may include a water electrolysis device, a CO₂ electrolysis device, a fuel cell, an electrolytic cell, and a vanadium flow battery.

The ion conductive polymer manufactured through the compound according to the embodiments of the present disclosure may have excellent ionic conductivity.

The separator manufactured through the compound may have excellent ionic conductivity.

The ion conductive polymer and separator manufactured through the compound of the present disclosure may be widely applied to green technology fields such as a water electrolysis device, a CO₂ electrolysis device and the like.

According to embodiments of the present disclosure, a method for manufacturing a monomer is provided, the method comprising a mixed solution preparation operation, and a coupling operation, the mixed solution preparation operation including a nitrogen bubbling operation for removing dissolved oxygen inside the mixed solution,
wherein the coupling operation includes use of an organic metal catalyst including an organometallic catalyst, a palladium catalyst, or a nickel catalyst for synthesizing the monomer of Formula 1,

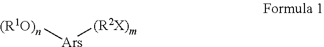

wherein in Formula 1, m and n are each independently an integer of 1 to 6, R¹ is each independently an organic group having 1 to 10 carbon atoms, R² is each independently an organic group having 1 to 20 carbon atoms, wherein X is a halogen atom, and Ars is a multi-aromatic ring having 10 to 50 carbon atoms.

According to an embodiment of the present disclosure, a compound including an organic group in which at least one hydrogen is substituted with a halogen atom, an ether group and a multi-aromatic ring is provided.

As the compound includes the organic group in which at least one hydrogen is substituted with a halogen atom, a quaternary ammonium functional group may be introduced into an ion conductive polymer manufactured from the compound. Accordingly, electrochemical properties of the ion conductive polymer may be improved.

As the compound includes the ether group, the compound may be polymerized under mild acidic conditions.

As the compound includes the multi-aromatic ring, it is possible to introduce the multi-aromatic ring into a main chain of the ion conductive polymer manufactured from the compound. Accordingly, the mechanical strength and chemical resistance of the ion conductive polymer may be improved.

### BRIEF DESCRIPTION OF THE DRAWINGS

FIG. 1 is a flow chart of a method for manufacturing a monomer according to exemplary embodiments.

### DETAILED DESCRIPTION

Hereinafter, the embodiments of the present disclosure will be described in detail with reference to the accompanying drawings. However, the embodiments are merely illustrative and are not limited to the specific embodiments described by way of illustration.

The term “organic group” as used herein may be a substituent which includes only carbon and hydrogen, or includes one or more atoms other than carbon and hydrogen. For example, the atoms other than carbon and hydrogen may include, for example, nitrogen (N), oxygen (O), phosphorus (P), and sulfur(S).

The term “multi-aromatic ring” as used herein may mean a compound which includes two or more aromatic rings. For example, the multi-aromatic ring may mean a compound in which compounds having two or more aromatic rings are consecutively linked, such as, for example, naphthalene, phenanthrene, and the like or a compound in which compounds having two or more aromatic rings are linked by a sigma (σ) bond, such as, for example, biphenyl, terphenyl, quaterphenyl, and 2,7-bisphenylfluorene.

The compound according to the embodiments of the present disclosure is represented by Formula 1 below.

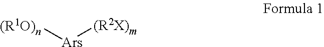

According to an embodiment, m may be an integer of 1 to 6, 1 to 5, or 1 to 3. For example, m may be 2.

If m is 7 or more, ammonium (NH₄⁺) may be excessively introduced into the ion conductive polymer manufactured by polymerization of the compound, thereby causing a decrease in the mechanical properties of the film.

According to an embodiment, n may be an integer of 1 to 6, 1 to 5, or 1 to 3. For example, n may be 2.

If n is 7 or more, ionic conductivity of the ion conductive polymer manufactured by polymerization of the compound may be reduced.

According to an embodiment, R¹ may each independently be an organic group having 1 to 10 carbon atoms, 1 to 8 carbon atoms, 1 to 6 carbon atoms, or 1 to 3 carbon atoms. For example, the R¹ may be a methyl group (—CH₃). Accordingly, the synthesis of the precursor polymer to be described below may be performed under relatively mild acidic conditions.

If the number of carbon atoms is 11 or more, the ionic conductivity of the ion conductive polymer manufactured by polymerization of the compound may be reduced.

According to an embodiment, R² may each independently be an organic group having 1 to 20 carbon atoms, 2 to 20 carbon atoms, 3 to 20 carbon atoms, 5 to 20 carbon atoms, or 6 to 20 carbon atoms, which includes at least one of an alkylene group or an arylene group. Accordingly, the quaternary ammonium functional group introduced into the ion conductive polymer manufactured by polymerization of the compound and the multi-aromatic rings included in the main chain may not be directly linked, thereby improving stability of the ion conductive polymer.

According to an embodiment, the X is a halogen atom. For example, X may be fluorine (F), chlorine (Cl), bromine (Br), or iodine (I), etc. Accordingly, a quaternary ammonium functional group may be introduced into the ion conductive polymer to be described below.

For example, X may each independently be chlorine (Cl), bromine (Br), or iodine (I). In one embodiment, X may each independently be bromine (Br), or iodine (I). In one embodiment, X may be iodine (I). The larger the particle size of the halogen atom, the easier it is to introduce a quaternary ammonium functional group into the ion conductive polymer manufactured by polymerization of the compound.

According to an embodiment, the Ars may be a multi-aromatic ring having 10 to 50 carbon atoms, 11 to 45 carbon atoms, 12 to 40 carbon atoms, or 15 to 30 carbon atoms. Accordingly, the compound may introduce the multi-aromatic ring into the main chain of the ion conductive polymer manufactured by polymerization.

If the number of carbon atoms in Ars exceeds 50, the solubility of the compound in a solvent may be greatly reduced, thereby resulting in low polymerization efficiency.

According to an embodiment, the compound represented by Formula 1 above may include at least one of compounds represented by Formulas 2 to 4 below.

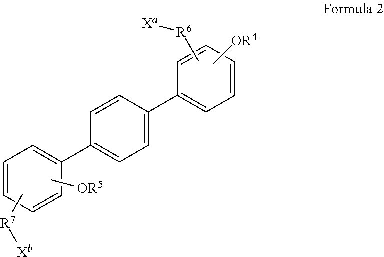

According to an embodiment, R⁴ and R⁵ may each independently be an organic group having 1 to 10 carbon atoms, 1 to 8 carbon atoms, 1 to 6 carbon atoms, or 1 to 3 carbon atoms. For example, the R⁴ and R⁵ may be a methyl group (—CH₃).

According to an embodiment, the R⁶ and R⁷ may each independently be an organic group having 1 to 20 carbon atoms, 2 to 20 carbon atoms, 3 to 20 carbon atoms, 5 to 20 carbon atoms, or 6 to 20 carbon atoms, which includes at least one of an alkylene group or an arylene group.

According to an embodiment, the X^{a} and X^{b} are halogen atoms. For example, X^{a} and X^{b} may each independently be fluorine (F), chlorine (Cl), bromine (Br), or iodine (I), etc. For example, X^{a} and X^{b} may each independently be chlorine (Cl), bromine (Br), or iodine (I). In one embodiment, X^{a} and X^{b} may each independently be bromine (Br) and iodine (I). In one embodiment, both X^{a} and X^{c} may be iodine (I).

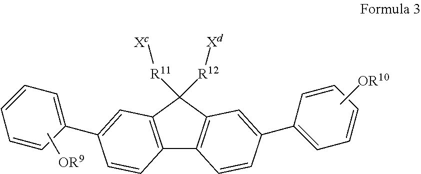

According to an embodiment, R⁹ and R¹⁰ may each independently be an organic group having 1 to 10 carbon atoms, 1 to 8 carbon atoms, 1 to 6 carbon atoms, or 1 to 3 carbon atoms. For example, the R⁹ and R¹⁰ may be a methyl group (—CH₃).

According to an embodiment, the R¹¹ and R¹² may each independently be an organic group having 1 to 20 carbon atoms, 2 to 20 carbon atoms, 3 to 20 carbon atoms, 5 to 20 carbon atoms, or 6 to 20 carbon atoms, which includes at least one of an alkylene group or an arylene group.

According to an embodiment, the X^{c} and X^{d} are halogen atoms. For example, X^{c} and X^{d} may each independently be fluorine (F), chlorine (Cl), bromine (Br), or iodine (I), etc. For example, X^{c} and X^{d} may each independently be chlorine (Cl), bromine (Br), or iodine (I). In one embodiment, X^{c} and X^{d} may each independently be bromine (Br), or iodine (I). In one embodiment, both X^{c} and X^{d} may be iodine (I).

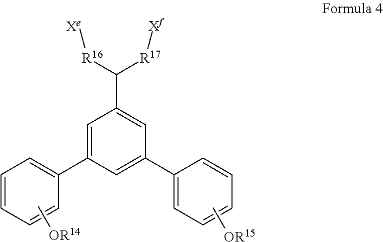

According to an embodiment, R¹⁴ and R¹⁵ may each independently be an organic group having 1 to 10 carbon atoms, 1 to 8 carbon atoms, 1 to 6 carbon atoms, or 1 to 3 carbon atoms. For example, the R¹⁴ and R¹⁵ may be a methyl group (—CH₃).

According to an embodiment, the R¹⁶ and R¹⁷ may each independently be an organic group having 1 to 20 carbon atoms, 2 to 20 carbon atoms, 3 to 20 carbon atoms, 5 to 20 carbon atoms, or 6 to 20 carbon atoms, which includes at least one of an alkylene group or an arylene group.

According to an embodiment, the X^{e} and X^{f} are halogen atoms. For example, X^{e} and X^{f} may each independently be fluorine (F), chlorine (Cl), bromine (Br), or iodine (I), etc. For example, X^{e} and X^{f} may each independently be chlorine (Cl), bromine (Br), or iodine (I). In one embodiment, X^{e} and X^{f} may each independently be bromine (Br), or iodine (I). In one embodiment, both X^{e} and XI may be iodine (I).

According to an embodiment, the compounds represented by Formulas 2 to 4 may be point symmetric or line symmetric with respect to a center point or center line (for example, middle phenyl among triphenyls in the case of Formula 2-1 or 4-1, or a center of cyclopentadiene or an imaginary center line passing through the center in the case of Formula 3-1) corresponding to a center of the compound. Accordingly, since both sides based on the center have the same reactivity, the synthesis and polymerization of the compound may be facilitated.

According to an embodiment, the compound represented by Formula 2 above may include a compound represented by Formula 2-1 below.

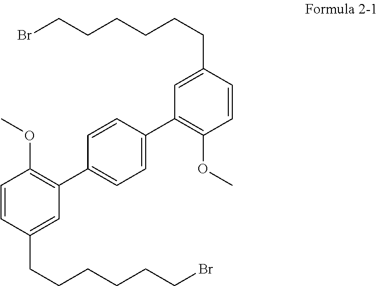 [SMILES: COc1ccc(CCCCCCBr)cc1-c1ccc(-c2cc(CCCCCCBr)ccc2OC)cc1]

According to an embodiment, the compound represented by Formula 3 above may include a compound represented by Formula 3-1 below.

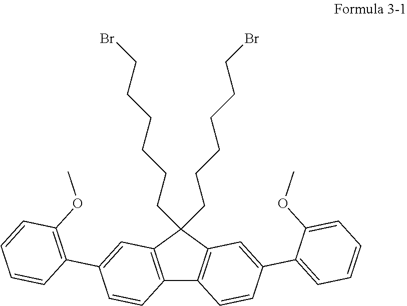 [SMILES: COc1ccccc1-c1ccc2c(c1)C(CCCCCCBr)(CCCCCCBr)c1cc(-c3ccccc3OC)ccc1-2]

According to an embodiment, the compound represented by Formula 4 above may include a compound represented by Formula 4-1 below.

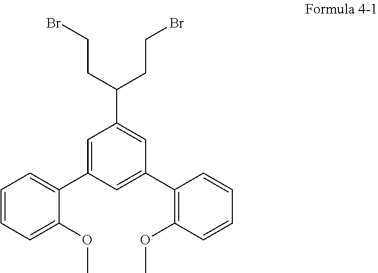 [SMILES: COc1ccccc1-c1cc(-c2ccccc2OC)cc(C(CCBr)CCBr)c1]

According to an embodiment, the compound represented by Formula 1 above may be a monomer for polymerization (hereinafter, may be abbreviated as a monomer). For example, the compound represented by Formula 1 above may be polymerized to become a precursor polymer of an ion conductive polymer (hereinafter, may be abbreviated as a precursor polymer).

According to an embodiment, the polymerization reaction may occur through a dehydration condensation reaction in the presence of an acidic catalyst. For example, the polymerization reaction may be a Fridel-Crafts polycondensation of the compound represented by Formula 1 above with an aldehyde represented by R³—CHO.

The precursor polymer according to embodiments of the present disclosure may be manufactured through polymerization of the monomer represented by Formula 1 above.

According to an embodiment, the precursor polymer may include a repeating unit represented by Formula 1a below.

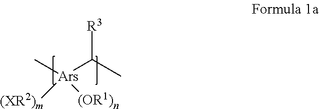

According to an embodiment, m may be an integer of 1 to 6, 1 to 5, or 1 to 3. For example, m may be 2.

If m is 7 or more, ammonium (NH₄⁺) may be excessively introduced into the ion conductive polymer manufactured from the precursor, thereby causing a decrease in the mechanical properties of the film.

According to an embodiment, n may be an integer of 1 to 6, 1 to 5, or 1 to 3. For example, n may be 2.

If n is 7 or more, ionic conductivity of the ion conductive polymer manufactured from the precursor may be reduced.

According to an embodiment, R¹ may each independently be an organic group having 1 to 10 carbon atoms, 1 to 8 carbon atoms, 1 to 6 carbon atoms, or 1 to 3 carbon atoms. For example, the R¹ may be a methyl group (—CH₃).

If the number of carbon atoms is 11 or more, the ionic conductivity of the ion conductive polymer manufactured from the precursor may be reduced.

According to an embodiment, R² may each independently be an organic group having 1 to 20 carbon atoms, 2 to 20 carbon atoms, 3 to 20 carbon atoms, 5 to 20 carbon atoms, or 6 to 20 carbon atoms, which includes at least one of an alkylene group or an arylene group. Accordingly, the quaternary ammonium functional group introduced into the ion conductive polymer to be described below and the multi-aromatic ring included in the main chain may not be directly linked, such that the stability of the ion conductive polymer may be improved.

According to an embodiment, the R³ may be H or an organic group having 1 to 15 carbon atoms, 1 to 10 carbon atoms, or 1 to 8 carbon atoms. For example, the R³ may be H. Accordingly, the synthesis of the precursor polymer may be facilitated.

According to an embodiment, when the R³ is an organic group other than H, the organic group may be further substituted with an electron withdrawing group (EWG). The electron withdrawing group may be, for example, a nitro group, a trifluoromethyl group, a cyano group, a fluoro group, an acyl group, an alkylsulfonyl group, etc. Accordingly, even when R³ is an organic group other than H, the synthesis of the precursor polymer may be facilitated.

According to an embodiment, the X is a halogen atom. For example, X may be fluorine (F), chlorine (Cl), bromine (Br), or iodine (I), etc. Accordingly, a quaternary ammonium functional group may be introduced into the ion conductive polymer to be described below.

For example, X may each independently be chlorine (Cl), bromine (Br), or iodine (I). In one embodiment, X may each independently be bromine (Br) and iodine (I). In one embodiment, X may be iodine (I). The larger the particle size of the halogen atom, the easier it is to introduce a quaternary ammonium functional group into the ion conductive polymer to be described below.

According to an embodiment, the Ars may be a multi-aromatic ring having 10 to 50 carbon atoms, 11 to 45 carbon atoms, 12 to 40 carbon atoms, or 15 to 30 carbon atoms. Accordingly, the multi-aromatic rings may be positioned in the main chain of the ion conductive polymer to be described below.

According to an embodiment, a repeating unit represented by Formula 1a above may include at least one of repeating units represented by Formulas 2a to 4a below.

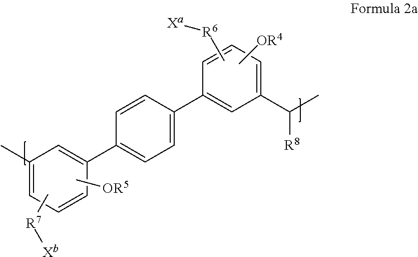

According to an embodiment, R⁴ and R⁵ may each independently be an organic group having 1 to 10 carbon atoms, 1 to 8 carbon atoms, 1 to 6 carbon atoms, or 1 to 3 carbon atoms. For example, the R⁴ and R⁵ may be a methyl group (—CH₃).

According to an embodiment, the R⁶ and R⁷ may each independently be an organic group having 1 to 20 carbon atoms, 2 to 20 carbon atoms, 3 to 20 carbon atoms, 5 to 20 carbon atoms, or 6 to 20 carbon atoms, which includes at least one of an alkylene group or an arylene group.

According to an embodiment, the R⁸ may be H or an organic group having 1 to 15 carbon atoms, 1 to 10 carbon atoms, or 1 to 8 carbon atoms.

According to an embodiment, the X^{a} and X^{b} are halogen atoms. For example, X^{a} and X^{b} may each independently be fluorine (F), chlorine (Cl), bromine (Br), or iodine (I), etc. For example, X^{a} and X^{b} may each independently be chlorine (Cl), bromine (Br), or iodine (I). In one embodiment, X^{a} and X^{b} may each independently be bromine (Br), or iodine (I). In one embodiment, both X^{a} and X^{c} may be iodine (I).

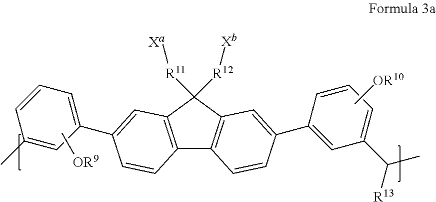

According to an embodiment, R⁹ and R¹⁰ may each independently be an organic group having 1 to 10 carbon atoms, 1 to 8 carbon atoms, 1 to 6 carbon atoms, or 1 to 3 carbon atoms. For example, the R⁹ and R¹⁰ may be a methyl group (—CH₃).

According to an embodiment, the R¹¹ and R¹² may each independently be an organic group having 1 to 20 carbon atoms, 2 to 20 carbon atoms, 3 to 20 carbon atoms, 5 to 20 carbon atoms, or 6 to 20 carbon atoms, which includes at least one of an alkylene group or an arylene group.

According to an embodiment, the R¹³ may be H or an organic group having 1 to 15 carbon atoms, 1 to 10 carbon atoms, or 1 to 8 carbon atoms.

According to an embodiment, the X^{c} and X^{d} are halogen atoms. For example, X^{c} and X^{d} may each independently be fluorine (F), chlorine (Cl), bromine (Br), or iodine (I), etc. For example, X^{c} and X^{d} may each independently be chlorine (Cl), bromine (Br), or iodine (I). In one embodiment, X^{c} and X^{d} may each independently be bromine (Br), or iodine (I). In one embodiment, both X^{c} and X^{d} may be iodine (I).

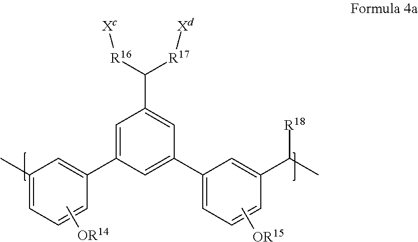

According to an embodiment, R¹⁴ and R¹⁵ may each independently be an organic group having 1 to 10 carbon atoms, 1 to 8 carbon atoms, 1 to 6 carbon atoms, or 1 to 3 carbon atoms. For example, the R¹⁴ and R¹⁵ may be a methyl group (—CH₃).

According to an embodiment, the R¹⁶ and R¹⁷ may each independently be an organic group having 1 to 20 carbon atoms, 2 to 20 carbon atoms, 3 to 20 carbon atoms, 5 to 20 carbon atoms, or 6 to 20 carbon atoms, which includes at least one of an alkylene group or an arylene group.

According to an embodiment, the R¹⁸ may be H or an organic group having 1 to 15 carbon atoms, 1 to 10 carbon atoms, or 1 to 8 carbon atoms.

According to an embodiment, the X^{e} and X^{f} are halogen atoms. For example, X^{e} and X^{f} may each independently be fluorine (F), chlorine (Cl), bromine (Br), or iodine (I), etc. For example, X^{e} and X^{f} may each independently be chlorine (Cl), bromine (Br), or iodine (I). In one embodiment, X^{e} and X^{f} may each independently be bromine (Br), or iodine (I). In one embodiment, both X^{e} and X may be iodine (I).

According to an embodiment, the repeating unit represented by Formula 2a above may include a repeating unit represented by Formula 2a-1 below.

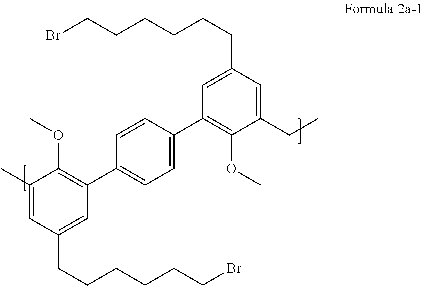

According to an embodiment, the repeating unit represented by Formula 3a above may include a repeating unit represented by Formula 3a-1 below.

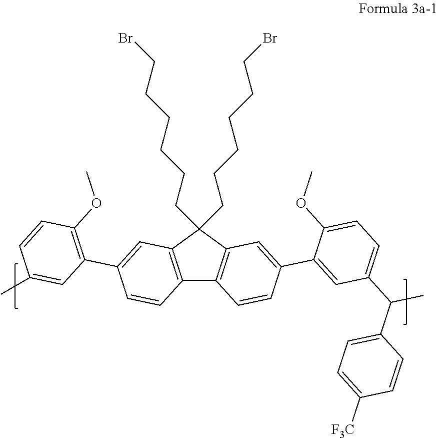

According to an embodiment, the repeating unit represented by Formula 4a above may include a repeating unit represented by Formula 4a-1 below.

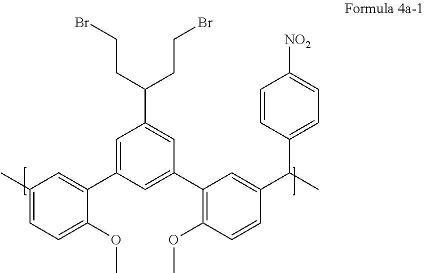

The ion conductive polymer according to embodiments of the present disclosure may be manufactured from a precursor polymer including the repeating unit represented by Formula 1a above.

For example, the ion conductive polymer may be manufactured through a bimolecular nucleophilic substitution reaction (S_{N}2 reaction) between the precursor polymer including the repeating unit represented by Formula 1a above and a tertiary amine compound.

As the tertiary amine compound, N,N-dimethylformamide, trimethylamine, triethylamine, tripropylamine, tributylamine, imidazole, N-methylpiperidine, quinuclidine, and the like may be used. According to an embodiment, the ion conductive polymer may include a repeating unit represented by Formula 5 below.

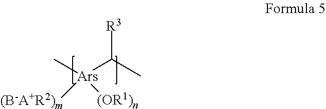

According to an embodiment, A⁺ may be a quaternary ammonium. According to an embodiment, A⁺ may be a quaternary ammonium including at least one methyl group.

In the present disclosure, the quaternary ammonium may also include ammonium in which two different substituents among substituents bonded to a nitrogen atom are linked to form a ring structure. For example, A⁺ may be trimethylammonium, triethylammonium, tripropylammonium, tributylammonium, imidazolium, N-methylpiperidinium, quinuclidinium and the like.

According to an embodiment, B⁻ may be an anion. According to an embodiment, B⁻ may be a monovalent or divalent anion. For example, when B⁻ is a divalent anion, two A⁺ and A⁺ located on different chains may bind together to one B⁻ to form a cross-linked structure.

According to an embodiment, B⁻ may be a halogen anion or a polyatomic anion. For example, B⁻ may be a chloride anion (Cl⁻), a bromine anion (Br⁻), an iodine anion (I⁻), a hydroxide ion (OH⁻), a sulfate anion (SO₄²⁻), a carbonate anion (CO₃²⁻), a bicarbonate anion (HCO₃⁻), or a carboxylic acid anion (RCO₂⁻).

According to an embodiment, m may be an integer of 1 to 6, 1 to 5, or 1 to 3. For example, m may be 2.

If m is 7 or more, mechanical properties of a film made of the ion conductive polymer may be decreased.

According to an embodiment, n may be an integer of 1 to 6, 1 to 5, or 1 to 3. For example, n may be 2.

If n is 7 or more, the ionic conductivity of the ion conductive polymer may be reduced.

According to an embodiment, the R¹ may be an organic group having 1 to 10 carbon atoms, 1 to 8 carbon atoms, 1 to 6 carbon atoms, or 1 to 3 carbon atoms. For example, the R¹ may be a methyl group (—CH₃).

If the number of carbon atoms is 11 or more, the ionic conductivity of the ion conductive polymer may be reduced.

According to an embodiment, in the ion conductive polymer including the repeating unit represented by the Formula 5 above, at least one of the hydrogens of a benzene ring may be substituted with an ether functional group. When the benzene ring of the ion conductive polymer and the ether functional group are bonded, the benzene ring is placed in an electron-rich state. Accordingly, the synthesis of the ion conductive polymer may be performed under relatively mild acidic conditions rather than super-strong acidic conditions such as triflic acid, which is also known as trifluoromethanesulfonic acid.

According to an embodiment, the mild acid may have an acid dissociation constant (pKa) of-7 or more.

For example, the mild acid may be methanesulfonic acid, trifluoroacetic acid, nitric acid, sulfuric acid, hydrochloric acid and the like.

According to an embodiment, the R² may be an organic group having 1 to 20 carbon atoms, 2 to 20 carbon atoms, 3 to 20 carbon atoms, 5 to 20 carbon atoms, or 6 to 20 carbon atoms, which includes at least one of an alkylene group or an arylene group.

In the repeating unit represented by Formula 5, if the number of carbon atoms between the quaternary ammonium ion and the benzene ring is too small, the ammonium functional group may be decomposed which in turn may decrease the mechanical strength and ionic conductivity of the ion conductive polymer.

According to embodiments of the present disclosure, since the quaternary ammonium ion is linked to the benzene ring through R², the electron withdrawing inductive and resonance effects of the benzene ring are reduced, such that the attack by OH may be decreased. Accordingly, the embodiments provide an effective solution that prevents decomposition of the ammonium functional group.

According to an embodiment, the R² may include both aromatic and aliphatic chains mixed together which may further improve the mechanical strength of the ion conductive polymer.

According to an embodiment, the R³ may be H or an organic group having 1 to 15, 1 to 10, or 1 to 8 carbon atoms. For example, the R³ may be H.

According to an embodiment, when the R³ is an organic group other than H, the organic group may be further substituted with an electron withdrawing group (EWG). The electron withdrawing group may be, for example, a nitro group (—NO₂), a trifluoromethyl group (—CF₃), a cyano group (—CN), a fluoro group (—F), an acyl group (—COR, where R is an organic substituent), an alkylsulfonyl group (—SO₂R, where R is an alkyl group), and the like.

According to an embodiment, the Ars may be a multi-aromatic ring having 10 to 50 carbon atoms, 11 to 45 carbon atoms, 12 to 40 carbon atoms, or 15 to 30 carbon atoms.

As the main chain of the ion conductive polymer includes the multi-aromatic ring, the mechanical strength and chemical resistance of the ion conductive polymer may be improved.

According to an embodiment, the repeating unit represented by Formula 5 above may include at least one of the repeating units represented by Formulas 6 to 8 below.

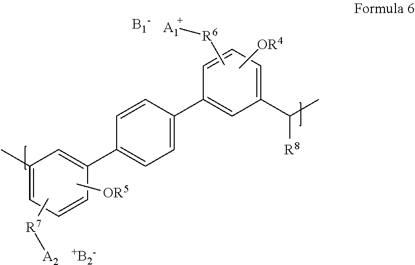

According to an embodiment, A₁⁺ and A₂⁺ may be quaternary ammoniums. According to an embodiment, A₁⁺ and A₂⁺ may be quaternary ammoniums including at least one methyl group.

According to an embodiment, B₁⁻ and B₂⁻ may be anions. For example, B₁⁻ and B₂⁻ may each independently be a chloride anion (Cl⁻), a bromine anion (Br⁻), an iodine anion (I⁻), a hydroxide ion (OH⁻), a sulfate anion (SO₄²⁻), a carbonate anion (CO₃²⁻), a bicarbonate anion (HCO₃⁻), or a carboxylic acid anion (RCO₂⁻).

According to an embodiment, the R⁴ and R⁵ may each independently be an organic group having 1 to 10 carbon atoms, 1 to 8 carbon atoms, 1 to 6 carbon atoms, or 1 to 3 carbon atoms. For example, the R⁴ and R⁵ may be methyl groups (—CH₃).

According to an embodiment, the R⁶ and R⁷ may each independently be an organic group having 1 to 20 carbon atoms, 1 to 18 carbon atoms, 1 to 15 carbon atoms, or 1 to 10 carbon atoms, which includes at least one of an alkylene group or an arylene group.

According to an embodiment, the R⁸ may be H or an organic group having 1 to 15 carbon atoms, 1 to 10 carbon atoms, or 1 to 8 carbon atoms.

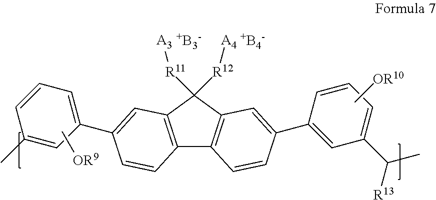

According to an embodiment, A₃⁺ and A₄⁺ may be quaternary ammoniums. According to an embodiment, A₃⁺ and A₄⁺ may be quaternary ammoniums including at least one methyl group.

According to an embodiment, B₃⁻ and B₄⁻ may be anions. For example, B₃⁻ and B₄⁻ may each independently be a chloride anion (Cl⁻), a bromine anion (Br⁻), an iodine anion (I⁻), a hydroxide ion (OH⁻), a sulfate anion (SO₄²⁻), a carbonate anion (CO₃²⁻), a bicarbonate anion (HCO₃⁻), or a carboxylic acid anion (RCO₂⁻).

According to an embodiment, R⁹ and R¹⁰ may each independently be an organic group having 1 to 10 carbon atoms, 1 to 8 carbon atoms, 1 to 6 carbon atoms, or 1 to 3 carbon atoms. For example, the R⁹ and R¹⁰ may be methyl groups (—CH₃).

According to an embodiment, the R¹¹ and R¹² may each independently be an organic group having 1 to 20 carbon atoms, 1 to 18 carbon atoms, 1 to 15 carbon atoms, or 1 to 10 carbon atoms, which includes at least one of an alkylene group or an arylene group.

According to an embodiment, the R¹³ may be H or an organic group having 1 to 15 carbon atoms, 1 to 10 carbon atoms, or 1 to 8 carbon atoms.

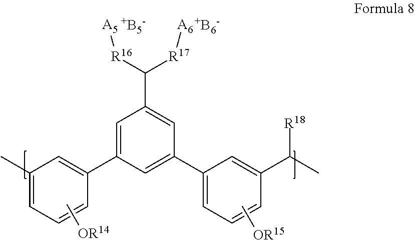

According to an embodiment, A₅⁺ and A₆⁺ may be quaternary ammoniums. According to an embodiment, A₅⁺ and A₆⁺ may be quaternary ammoniums including at least one methyl group.

According to an embodiment, B₅⁻ and B₆⁻ may be anions. For example, B₅⁻ and B₆⁻ may each independently be a chloride anion (Cl⁻), a bromine anion (Br⁻), an iodine anion (I⁻), a hydroxide ion (OH⁻), a sulfate anion (SO₄²⁻), a carbonate anion (CO₃²⁻), a bicarbonate anion (HCO₃⁻), or a carboxylic acid anion (RCO₂⁻).

According to an embodiment, R¹⁴ and R¹⁵ may each independently be an organic group having 1 to 10 carbon atoms, 1 to 8 carbon atoms, 1 to 6 carbon atoms, or 1 to 3 carbon atoms. For example, the R¹⁴ and R¹⁵ may be methyl groups (—CH₃).

According to an embodiment, the R¹⁶ and R¹⁷ may each independently be an organic group having 1 to 20 carbon atoms, 1 to 18 carbon atoms, 1 to 15 carbon atoms, or 1 to 10 carbon atoms, which includes at least one of an alkylene group or an arylene group.

According to an embodiment, the R¹⁸ may be H or an organic group having 1 to 15 carbon atoms, 1 to 10 carbon atoms, or 1 to 8 carbon atoms.

According to an embodiment, the repeating unit represented by Formula 6 above may include a repeating unit represented by Formula 6-1 below.

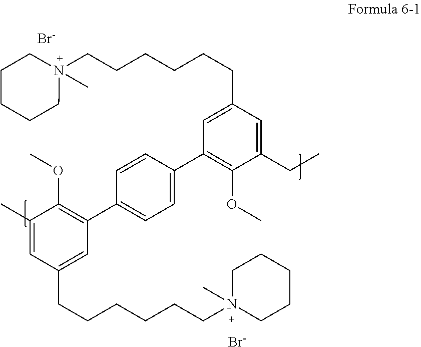

According to an embodiment, the repeating unit represented by Formula 7 above may include a repeating unit represented by Formula 7-1 below.

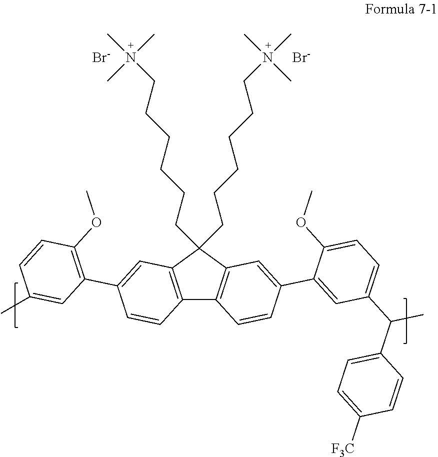

According to an embodiment, the repeating unit represented by Formula 8 above may include a repeating unit represented by Formula 8-1 below.

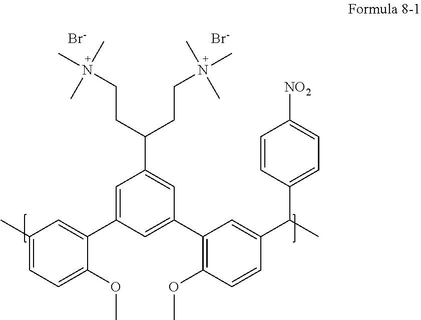

According to an embodiment, OH ionic conductivity of the ion conductive polymer at 25° C. may be 40 mS/cm to 80 mS/cm. For example, the OH ionic conductivity of the ion conductive polymer at 25° C. may be 43 mS/cm to 70 mS/cm, 45 mS/cm to 65 mS/cm, 46 mS/cm to 63 mS/cm, or 47 mS/cm to 60 mS/cm.

Hereinafter, a method for manufacturing a monomer according to embodiments of the present disclosure is provided.

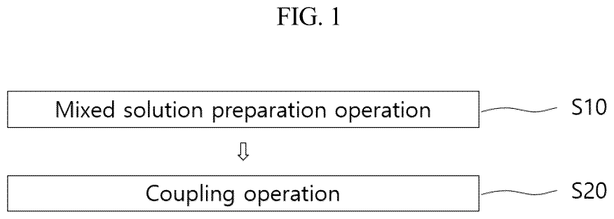

FIG. 1 is a flow chart of a method for manufacturing a monomer according to exemplary embodiments.

The method for manufacturing a monomer according to embodiments may include a mixed solution preparation operation S10 and a coupling operation S20.

The mixed solution preparation operation may include a nitrogen (N₂) bubbling operation. Accordingly, dissolved oxygen (O₂) inside the mixed solution may be removed.

The coupling operation may be performed through an organic metal catalyst. As the organometallic catalyst, a palladium catalyst, or a nickel catalyst, etc. may be used. Accordingly, the monomer represented by Formula 1 above may be synthesized. The monomer represented by Formula 1 above may include an alkoxy group and a multi-aromatic ring.

The alkoxy group may be —(OR¹)ₙ in Formula 1 above, and n may be an integer of 1 to 6.

The multi-aromatic ring may be bonded with —(R²A⁺B⁻)ₘ whose terminal is substituted with a halogen, and m may be an integer of 1 to 6.

The method for manufacturing an ion conductive polymer according to embodiments may include a monomer preparation operation S30, a precursor synthesis operation S40 and an ion conductive polymer synthesis operation S50.

The monomer used in the monomer preparation operation S30 may be obtained by purifying the monomer synthesized through the above-described monomer preparation method.

In the precursor synthesis operation S40, the monomer may be reacted with an aldehyde under mild acidic conditions to obtain a precursor polymer.

When reacting the monomer with aldehyde under mild acidic conditions (primary polymerization), the multi-aromatic ring of the monomer may be included in the main chain of the precursor polymer.

The primary polymerization may be carried out in the presence of a mild acid. As the mild acid, the above-described mild acids may be used. For example, an acid having a pKa of −7 or more may be used.

As described above, when at least one of the hydrogens of the benzene ring is substituted with an ether functional group, the benzene ring is placed in an electron-rich state. Accordingly, the synthesis of the ion conductive polymer may be performed under relatively mild acidic conditions rather than super-strong acidic conditions such as triflic acid.

The aldehyde may have R³—CHO, and after the primary polymerization, it will have a structure in which R³ is linked to the main chain of the precursor polymer.

In some embodiments, R³ may be further substituted with an electron withdrawing group, and in this case, the precursor polymer may be more easily produced by increasing the reactivity of the aldehyde.

In the ion conductive polymer synthesis operation S50, a quaternary ammonium group is introduced into the precursor polymer to synthesize an ion conductive polymer including the repeating unit represented by Formula 5 above.

When the quaternary ammonium group is introduced into a side chain of the multi-aromatic ring, the ion conductive polymer may exhibit ionic conductivity.

According to embodiments of the present disclosure, a separator including the ion conductive polymer is provided.

According to an embodiment, the separator may be manufactured by laminating ion conductive polymer films including the repeating unit represented by the Formula 5 above.

According to an embodiment, the separator may have a tensile strength of 10 MPa to 80 MPa, 15 MPa to 70 MPa, 20 MPa to 60 MPa, 25 MPa to 50 MPa. The tensile strength may be measured using a universal testing machine (UTM) in an environment of 25° C. and a relative humidity of 50%.

According to embodiments of the present disclosure, a device including the separator is provided. The device may include a cathode; an anode disposed to face the cathode; and the separator disposed between the cathode and the anode. The device according to embodiments may include a water electrolysis device, a CO₂ electrolysis device, a fuel cell, an electrolytic cell, a vanadium flow battery and the like. Hereinafter, examples are proposed to facilitate understanding of the embodiments of the present disclosure, but these examples are only given for illustrating the embodiments and are not intended to limit the appended claims. It will be apparent to those skilled in the art that various alterations and modifications are possible within the scope and spirit of the present disclosure, and such alterations and modifications are duly included in the appended claims. Furthermore, the embodiments may be combined to form additional embodiments.

### Preparative Example 1: Preparation of Monomer 1 (A-1)

### Example 1

20 mL of 1,2-dimethoxyethane, 20 mL of distilled water, 1,4-benzenediboronic acid (1.0 g, 6 mmol), 2-bromo-4 (6-bromohexyl)anisole (4.6 g, 13 mmol), and sodium carbonate (Na₂CO₃, 3.8 g, 36 mmol) were added to a 100 mL round bottom flask, followed by bubbling with nitrogen (N₂) for 30 minutes while stirring to prepare a mixed solution.

Tetrakis(triphenylphosphine)palladium(0) (Pd(PPh₃)₄, 418 mg, 0.4 mmol) was added to the prepared mixed solution while maintaining a nitrogen (N₂) atmosphere, and refluxed for 24 hours by increasing the temperature to 80° C. After refluxing, the mixture was cooled to room temperature, extracted twice with 20 mL of dichloromethane, and then moisture was removed with magnesium sulfate and concentrated under reduced pressure to obtain a solid.

The solid was purified by column chromatography to prepare 3.5 g of a compound of Example 1 represented by Formula 2-1 below as Monomer (A-1).

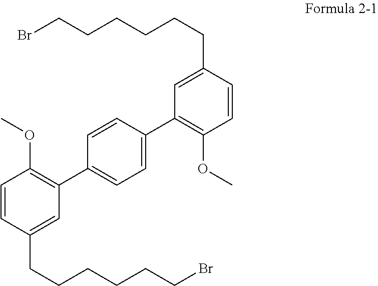 [SMILES: COc1ccc(CCCCCCBr)cc1-c1ccc(-c2cc(CCCCCCBr)ccc2OC)cc1]

Results of ¹H-nuclear magnetic resonance (¹H-NMR) spectroscopy analysis performed on the prepared A-1 are as follows.

¹H-NMR (CDCl₃, ppm): 7.57 (s, 4H), 7.19 (d, 2H), 7.12 (dd, 2H), 6.91 (d, 2H), 3.81 (s, 6H), 3.41 (t, 4H), 2.61 (t, 4H), 1.86 (m, 4H), 1.64 (m, 4H), 1.47 (m, 4H), 1.38 (m, 4H)

### Example 2: Preparation of Monomer 2 (A-2)

15 mL of 1,2-dimethoxyethane, 15 mL of distilled water, 2,7-dibromo-9,9-bis(6-bromohexyl)fluorine (3.0 g, 5 mmol), 2-methoxyphenylboronic acid (1.8 g, 12 mmol), and sodium carbonate (Na₂CO₃, 1.2 g, 12 mmol) were added to a 100 mL round bottom flask, followed by bubbling with nitrogen (N₂) for 30 minutes while stirring to prepare a mixed solution.

Tetrakis(triphenylphosphine)palladium(0) (Pd(PPh₃)₄, 267 mg, 0.2 mmol) was added to the prepared mixed solution while maintaining a nitrogen (N₂) atmosphere, and refluxed for 24 hours by increasing the temperature to 80° C. After refluxing, the mixture was cooled to room temperature, extracted twice with 20 mL of dichloromethane, and then moisture was removed with magnesium sulfate and concentrated under reduced pressure to obtain a solid.

The solid was purified by column chromatography to prepare 3.0 g of a compound of Example 2 represented by Formula 3-1 below as Monomer (A-2).

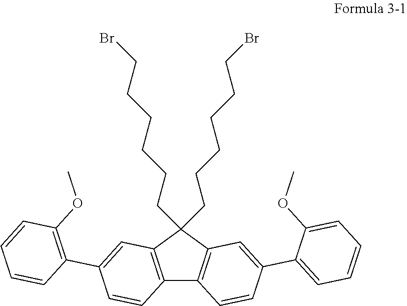 [SMILES: COc1ccccc1-c1ccc2c(c1)C(CCCCCCBr)(CCCCCCBr)c1cc(-c3ccccc3OC)ccc1-2]

Results of ¹H-nuclear magnetic resonance (¹H-NMR) spectroscopy analysis performed on the prepared A-2 are as follows.

¹H-NMR (CDCl₃, ppm): 7.74 (d, 2H), 7.52 (m, 4H), 7.41 (dd, 2H), 7.34 (m, 2H), 7.07 (t, 2H), 7.02 (d, 2H), 3.84 (s, 6H), 3.28 (t, 4H), 1.99 (m, 4H), 1.68 (m, 4H), 1.23 (m, 4H), 1.10 (m, 4H), 0.84 (m, 4H)

### Example 3: Preparation of Monomer 3 (A-3)

15 mL of 1,2-dimethoxyethane, 15 mL of distilled water, 1,3-dibromo-5-(1,5-dibromopentane-3-yl)benzene (3.0 g, 6 mmol), 2-methoxyphenylboronic acid (2.5 g, 16 mmol), and sodium carbonate (Na₂CO₃, 1.7 g, 16 mmol) were added to a 100 mL round bottom flask, followed by bubbling with nitrogen (N₂) for 30 minutes while stirring to prepare a mixed solution.

Tetrakis(triphenylphosphine)palladium(0) (Pd(PPh₃)₄, 374 mg, 0.3 mmol) was added to the prepared mixed solution while maintaining a nitrogen (N₂) atmosphere, and refluxed for 24 hours by increasing the temperature to 80° C. After refluxing, the mixture was cooled to room temperature, extracted twice with 20 mL of dichloromethane, and then moisture was removed with magnesium sulfate and concentrated under reduced pressure to obtain a solid.

The solid was purified by column chromatography to prepare 2.7 g of a compound of Example 3 represented by Formula 4-1 below as Monomer (A-3).

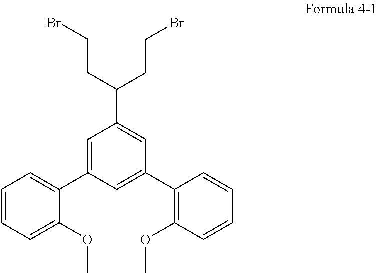 [SMILES: COc1ccccc1-c1cc(-c2ccccc2OC)cc(C(CCBr)CCBr)c1]

Results of ¹H-nuclear magnetic resonance (¹H-NMR) spectroscopy analysis performed on the prepared A-3 are as follows.

¹H-NMR (CDCl₃, ppm): 8.02 (m, 4H), 7.84 (s, 1H), 7.49 (dd, 2H), 7.14 (m, 4H), 3.79 (s, 6H), 3.52 (t, 4H), 2.58 (m, 1H), 2.06 (q, 4H)

### Preparative Example 2: Preparation of Precursor Polymer

### (1) Preparation of Precursor Polymer 1 (B-1) Using the Monomer of Example 1

20 mL of dichloromethane, 1.6 mL of methanesulfonic acid, paraformaldehyde (corresponding to 0.11 g, 3.6 mmol of formaldehyde), and the prepared A-1 (2 g, 3.2 mmol) were added to a 25 mL round bottom flask and stirred for 2 hours. After stirring, 200 mL of methanol was put therein to precipitate a solid. The precipitated solid was filtered and washed twice with 50 mL of methanol, then dried in an oven to prepare 1.8 g of Precursor Polymer 1 (B-1) represented by Formula 2a-1 below.

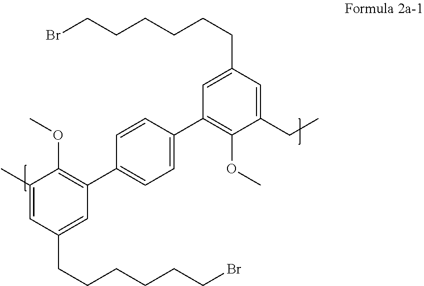

A weight average molar mass of the prepared B-1, measured by gel permeation chromatography (GPC) using polystyrene as a reference material, was 150,000 Da.

### (2) Preparation of Precursor Polymer 2 (B-2) Using the Monomer of Example 2

12 mL of dichloromethane, 1 mL of methanesulfonic acid, 4 (trifluoromethyl)benzaldehyde (0.99 g, 5.7 mmol), and the prepared A-2 (2 g, 2.8 mmol) were added to a 25 mL round bottom flask and stirred for 8 hours. After stirring, 200 mL of methanol was put therein to precipitate a solid. The precipitated solid was filtered and washed twice with 50 mL of methanol, then dried in an oven to prepare 2.3 g of Precursor Polymer 2 (B-2) represented by Formula 3a-1 below.

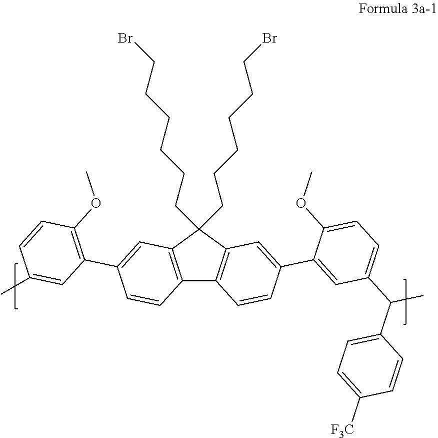

A weight average molar mass of the prepared B-2, measured by gel permeation chromatography (GPC) using polystyrene as a reference material, was 210,000 Da.

### (3) Preparation of Precursor Polymer 3 (B-3) Using the Monomer of Example 3

12 mL of dichloromethane, 3.7 mL of methanesulfonic acid, 4-nitrobenzaldehyde (0.7 g, 4.6 mmol), and the prepared A-3 (2 g, 3.9 mmol) were added to a 25 mL round bottom flask and stirred for 8 hours. After stirring, 200 mL of methanol was put therein to precipitate a solid. The precipitated solid was filtered and washed twice with 50 mL of methanol, then dried in an oven to prepare 2.4 g of Precursor Polymer 3 (B-3) represented by Formula 4a-1 below.

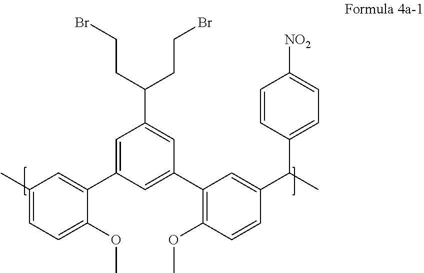

A weight average molar mass of the prepared B-3, measured by gel permeation chromatography (GPC) using polystyrene as a reference material, was 78,000 Da.

### Preparative Example 3: Manufacturing of Ion Conductive Polymer Film (Separator)

### (1) Manufacturing of Ion Conductive Polymer Film (Separator) (C-1) Using the Monomer of

### Example 1

2.2 mL of N,N-dimethylformamide and 0.3 g of the prepared B-1 were put into a 20 mL glass vial and stirred at room temperature (25° C.). After B-1 was completely dissolved, 0.12 g of N-methylpiperidine was added thereto and stirred at 80° C. for 24 hours. After stirring, the solution was poured into a petri dish and then dried in an oven at 80° C. for 24 hours to manufacture Separator (C-1) formed of a polymer including a repeating unit represented by Formula 6-1 below.

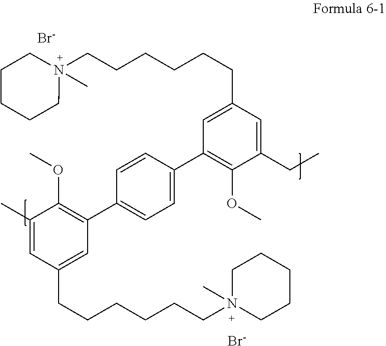

Results of ¹H-nuclear magnetic resonance (¹H-NMR) spectroscopy analysis performed on the prepared C-1 are as follows.

¹H-NMR (DMSO-do ppm): 7.72 (br, 2H), 7.25 (br, 4H), 7.23 (br, 2H), 3.99 (br, 2H), 3.79 (br, 6H), 3.30 (br, 6H), 3.22 (br, 12H), 2.64 (br, 4H), 1.71 (br, 12H), 1.61 (br, 8H), 1.29 (br, 8H)

### (2) Preparation of Ion Conductive Polymer Film (Separator) (C-2) Using the Monomer of Example 2

2.6 mL of N,N-dimethylformamide and 0.3 g of the prepared B-2 were put into a 20 mL glass vial and stirred at room temperature (25° C.). After B-2 was completely dissolved, 0.46 g of a trimethylamine aqueous solution (28%) was added thereto and stirred at room temperature (25° C.) for 24 hours. After stirring, the solution was poured into a petri dish and then dried in an oven at 80° C. for 24 hours to manufacture Separator (C-2) formed of a polymer including a repeating unit represented by Formula 7-1 below.

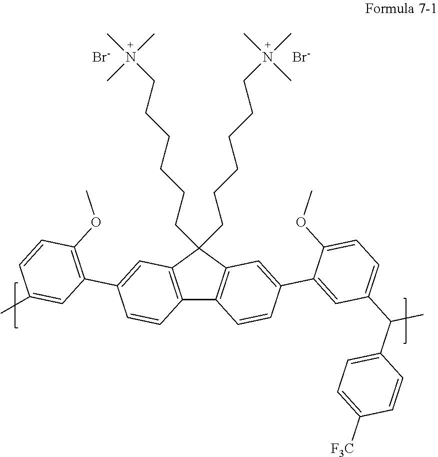

Results of ¹H-nuclear magnetic resonance spectroscopy analysis performed on the prepared C-2 are as follows.

¹H-NMR (DMSO-d₆, ppm): 7.85 (br, 2H), 7.75 (br, 2H), 7.57 (br, 2H), 7.51 (br, 2H), 7.44 (br, 2H), 7.28 (br, 2H), 7.16 (br, 4H), 5.91 (br, 1H), 3.81 (br, 6H), 3.57 (br, 18H), 3.20 (br, 4H), 1.97 (br, 4H), 1.47 (br, 4H), 1.06 (br, 8H), 0.69 (br, 4H)

### (3) Preparation of Ion Conductive Polymer Film (Separator) (C-3) Using the Monomer of Example 3

2.2 mL of N,N-dimethylformamide and 0.3 g of the prepared B-3 were put into a 20 mL glass vial and stirred at room temperature (25° C.). After B-3 was completely dissolved, 0.41 g of an aqueous trimethylamine solution (28%) was added thereto and stirred at room temperature (25° C.) for 24 hours. After stirring, the solution was poured into a petri dish and then dried in an oven at 80° C. for 24 hours to manufacture Separator (C-3) formed of a polymer including a repeating unit represented by Formula 8-1 below.

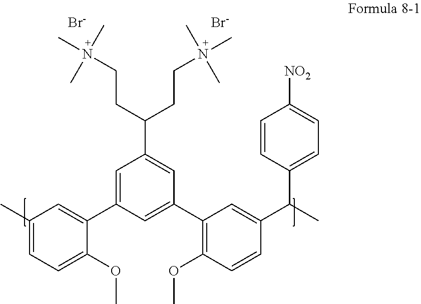

Results of ¹H-nuclear magnetic resonance spectroscopy analysis performed on the prepared C-3 are as follows.

¹H-NMR (DMSO-do, ppm): 8.16 (br, 2H), 8.04 (br, 2H), 7.80 (br, 3H), 7.44 (br, 2H), 7.21 (br, 2H), 7.14 (br, 2H), 5.41 (br, 1H), 3.79 (br, 6H), 3.30 (br, 18H), 3.22 (br, 4H), 2.58 (br, 1H), 2.00 (br, 4H)

### Comparative Example 1

Sustainion® X37-50 Grade RT product produced by Dioxide Materials was used.

### Comparative Example 2

Sustainion® X37-50 Grade T product produced by Dioxide Materials was used.

### Experimental Example 1: Evaluation of Ionic Conductivity of Ion Conductive Polymer Film (Separator)

The separators manufactured using the monomers of Examples 1 to 3 and the membrane samples of Comparative Examples 1 to 2 were cut into a size of 1 cm×3 cm and fixed between Pt electrodes of a Conductivity Clamp (BT-110, Scribner).

An ionic conductivity (σ) of an anion exchange membrane was evaluated in the form of OH⁻ under conditions of room temperature (25° C.) and tertiary distilled water. A membrane resistance (R) was measured by means of a 4-point probe method using an impedance analyzer (VSP-3e, Biologics) in a frequency range of 0.1 kHz to 1 MHz. A thickness (T) of the membrane sample was measured using a micrometer.

The ionic conductivity (σ) was calculated using Equation 1 below, and results thereof are shown in Table 1 below.

$$σ = \frac{L}{R \times A} = \frac{L}{R \times W \times T} \qquad Equation 1$$

In Equation 1, R is a membrane resistance (Ω), A is a cross-sectional area (cm²) of the membrane sample, L is a distance (cm) between electrodes, W is a width (cm) of the membrane sample, and T is a thickness (cm) of the membrane sample.

TABLE 1

| | OH⁻ ionic conductivity |
|---|---|
| | (mS/cm) |
| Separator manufactured using | 54.5 |
| monomer of Example 1 | |
| Separator manufactured using | 53.7 |
| monomer of Example 2 | |
| Separator manufactured using | 53.0 |
| monomer of Example 1 | |
| Separator of Comparative | 38.0 |
| Example 1 | |
| Separator of Comparative | 32.7 |
| Example 2 | |

Referring to Table 1, it can be confirmed that the OH ionic conductivities of the separators manufactured using the monomers of Examples 1 to 3 are superior to the OH ionic conductivities of the separators of Comparative Examples 1 to 2.

### Experimental Example 2: Evaluation of Tensile Strength of Ion Conductive Polymer Film (Separator)

The separator samples manufactured using the monomers of Examples 1 to 3 were cut into a size of 1 cm×6 cm, and tensile strengths were measured using a universal testing machine (Instron) at a crosshead speed of 10 mm/min in an environment of a temperature of 25° C. and a relative humidity of 50%, and results thereof are shown in Table 2 below.

TABLE 2

| | Tensile strength (MPa) |
|---|---|
| Separator manufactured using | 28.1 |
| monomer of Example 1 | |
| Separator manufactured using | 42.3 |
| monomer of Example 2 | |
| Separator manufactured using | 35.9 |
| monomer of Example 3 | |

Referring to Table 2, the tensile strengths of the separators manufactured using the monomers of Examples 1 to 3 were excellent at 28.1 MPa or more.

On the other hand, the strength of the separator of Comparative Example 1 was insufficient, thereby measurement of the tensile strength was impossible.

The separator of Comparative Example 2 corresponded to a reinforced film to have a tensile strength corresponding to the separators of the Examples, but as described above, the OH ionic conductivity was lower than that of the Examples.

What is claimed is:

1. A compound represented by Formula 1 below: 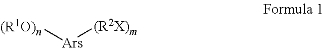
wherein in Formula 1, m and n are each independently an integer of 1 to 6, R¹ is each independently an organic group having 1 to 10 carbon atoms, R² is each independently an organic group having 1 to 20 carbon atoms, wherein the R² includes at least one of an alkylene group or an arylene group, X is a halogen atom, and Ars is a multi-aromatic ring having 10 to 50 carbon atoms.

2. The compound according to claim 1, wherein
the compound represented by Formula 1 above comprises at least one of compounds represented by Formulas 2 to 4 below: 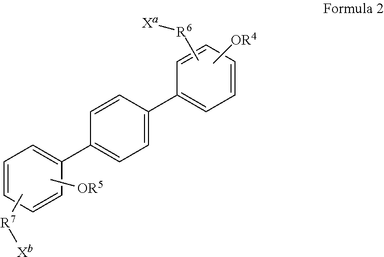
wherein in Formula 2, R⁴ and R⁵ are each independently an organic group having 1 to 10 carbon atoms, R⁶ and R⁷ are each independently an organic group having 1 to 20 carbon atoms, wherein the R⁶ and R⁷ include at least one of an alkylene group or an arylene group, and X^{a} and X^{b} are halogen atoms, 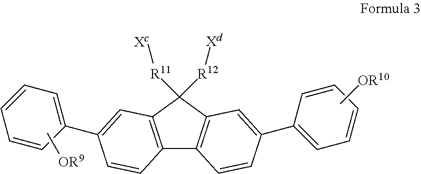
wherein in Formula 3, R⁹ and R¹⁰ are each independently an organic group having 1 to 10 carbon atoms, R¹¹ and R¹² are each independently an organic group having 1 to 20 carbon atoms, wherein the R¹¹ and R¹² include at least one of an alkylene group or an arylene group, and X^{c} and X^{d} are halogen atoms, 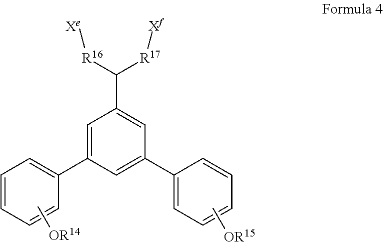
wherein in Formula 4, R¹⁴ and R¹⁵ are each independently an organic group having 1 to 10 carbon atoms, R¹⁶ and R¹⁷ are each independently an organic group having 1 to 20 carbon atoms, wherein the R¹⁶ and R¹⁷ include at least one of an alkylene group or an arylene group, and X^{e} and X^{f} are halogen atoms.

3. The compound according to claim 2, wherein
the compounds represented by Formulas 2 to 4 above include compounds represented by Formulas 2-1 to 4-1 below, respectively:

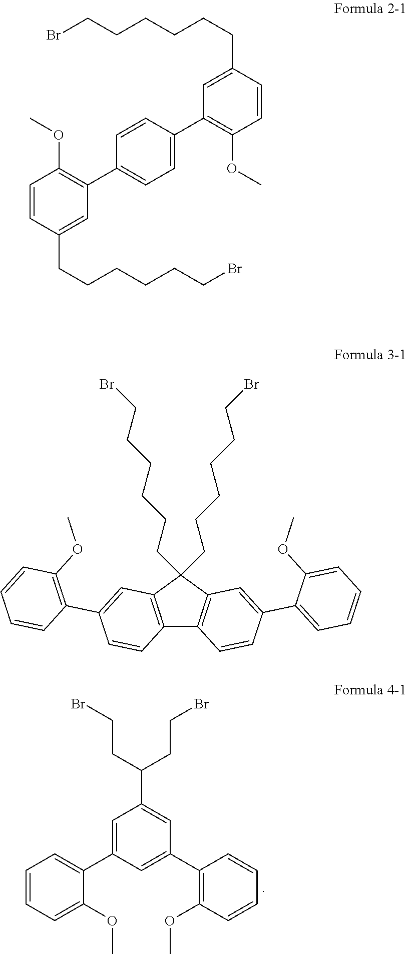 [SMILES: COc1ccc(CCCCCCBr)cc1-c1ccc(-c2cc(CCCCCCBr)ccc2OC)cc1 ; COc1ccccc1-c1ccc2c(c1)C(CCCCCCBr)(CCCCCCBr)c1cc(-c3ccccc3OC)ccc1-2 ; COc1ccccc1-c1cc(-c2ccccc2OC)cc(C(CCBr)CCBr)c1]

4. The compound according to claim 1, wherein
the compound is a monomer for polymerization.

5. An ion conductive polymer comprising a repeating unit represented by Formula 5 below: 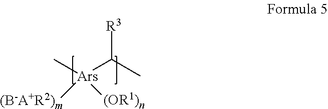
wherein in Formula 5, A⁺ is a quaternary ammonium, B⁻ is an anion, m and n are each independently an integer of 1 to 6, R¹ is each independently an organic group having 1 to 10 carbon atoms, R² is each independently an organic group having 1 to 20 carbon atoms wherein the R² includes at least one of an alkylene group or an arylene group, R³ is H or an organic group having 1 to 15 carbon atoms, and Ars is a multi-aromatic ring having 10 to 50 carbon atoms.

6. The ion conductive polymer according to claim 5, wherein
the repeating unit represented by Formula 5 above comprises at least one of repeating units represented by Formulas 6 to 8 below: 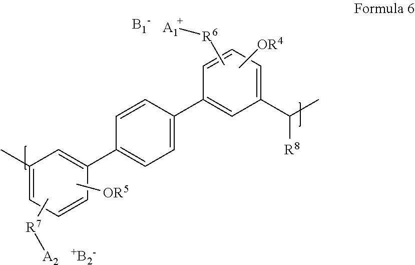
wherein in Formula 6, A₁⁺ and A₂⁺ are quaternary ammoniums, B₁⁻ and B₂⁻ are anions, R⁴ and R⁵ are each independently an organic group having 1 to 10 carbon atoms, R⁶ and R⁷ are each independently an organic group having 1 to 20 carbon atoms, wherein the R⁶ and R⁷ include at least one 5 of an alkylene group or an arylene group, and R⁸ is H or an organic group having 1 to 15 carbon atoms, 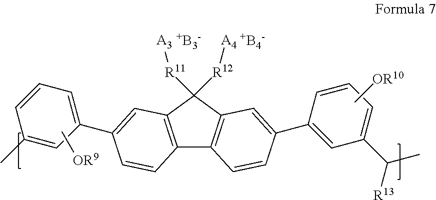
wherein in Formula 7, A₃⁺ and A₄⁺ are quaternary ammoniums, B₃⁻ and B₄⁻ are anions, R⁹ and R¹⁰ are each independently an organic group having 1 to 10 carbon atoms, R¹¹ and R¹² are each independently an organic group having 1 to 20 carbon atoms, wherein the R¹¹ and R¹² include at least one of an alkylene group or an arylene group, and R¹³ is H or an organic group having 1 to 15 carbon atoms, 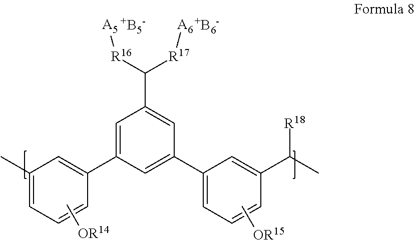
wherein in Formula 8, A₅⁺ and A₆⁺ are quaternary ammoniums, B₅⁻ and B₆⁻ are anions, R¹⁴ and R¹⁵ are each independently an organic group having 1 to 10 carbon atoms, R¹⁶ and R¹⁷ are each independently an organic group having 1 to 20 carbon atoms, wherein the R¹⁶ and R¹⁷ include at least one of an alkylene group or an arylene group, and R¹⁸ is H or an organic group having 1 to 15 carbon atoms.

7. The ion conductive polymer according to claim 6, wherein
the repeating units represented by Formulas 6 to 8 above include repeating units represented by Formulas 6-1 to 8-1 below, respectively:

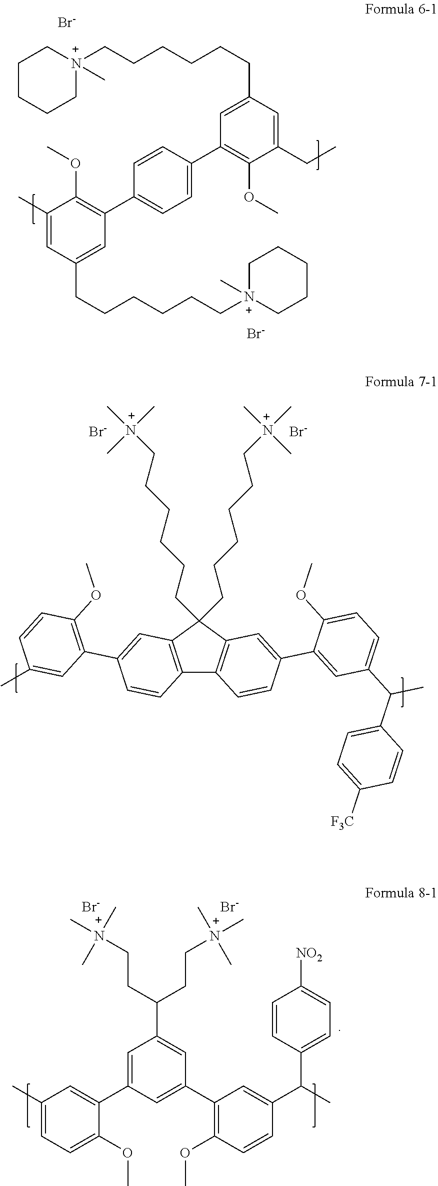

8. The ion conductive polymer according to claim 5, wherein
an OH⁻ ionic conductivity of the ion conductive polymer at 25° C. is 40 mS/cm to 80 mS/cm.

9. A device comprising: a cathode;
an anode disposed to face the cathode; and
the separator of claim 5.

10. The device according to claim 9, wherein the device comprises a water electrolysis device, a CO₂ electrolysis device, a fuel cell, an electrolytic cell, and a vanadium flow battery.
